(19) United States
(12) Patent Application Publication
(10) Pub. No.: US 2022/0313774 A1
(43) Pub. Date: Oct. 6, 2022

(54) INTRANASAL ADMINISTRATION OF MEROTOCIN FOR IMPROVING LACTATION

(71) Applicant: FERRING B.V., Hoofddorp (NL)

(72) Inventors: Deborah ARBIT, Parsippany, NJ (US); Joan-Carles ARCE SAEZ, Parsippany, NJ (US)

(73) Assignee: FERRING B.V., Hoofddorp (NL)

(21) Appl. No.: 17/618,416

(22) PCT Filed: Nov. 3, 2020
(86) PCT No.: PCT/EP2020/080831

§ 371 Date: Dec. 10, 2021

Related U.S. Application Data
(60) Provisional application No. 62/930,427, filed on Nov. 4, 2019.

(30) Foreign Application Priority Data
Mar. 18, 2020 (DK) PA 2020 00349

(51) Int. Cl. A61K 38/095; A61K 9/00; A61P 15/14; A61K 47/12; A61K 47/02

(52) U.S. Cl. CPC A61K 38/095; A61K 9/0043; A61P 15/14; A61K 47/12; A61K 47/02

(57) ABSTRACT

The present disclosure relates to the use of a novel dosage form of merotocin in improving lactation in a female in need thereof. The disclosure particularly provides the use of a composition suitable for intranasal administration that provides a dose of merotocin greater than 100 μg or substantially about 400 μg to the female. The disclosure encompasses methods of treatment as well as compositions for use in such methods.

### FIELD

The present disclosure relates to the use of merotocin in improving lactation in a female in need thereof. In particular, the merotocin may be provided in the form of a composition for intranasal administration. The disclosure particularly provides the use of a composition comprising greater than 100 μg, for example substantially about 400 μg, merotocin in improving lactation in a female in need thereof (e.g. providing a dose of merotocin greater than 100 μg or substantially about 400 μg to the female). The disclosure encompasses methods of treatment as well as compositions for use in such methods.

### BACKGROUND

Breastfeeding is known to be beneficial for both mothers and infants. However, despite measures to support and increase breastfeeding rates, many women experience lactation difficulties.

Mother's own milk (MoM) is also generally recognized as the preferred option for the health and development of a preterm infant. For example, numerous studies have shown that bovine-based formula milk causes pro-inflammatory changes in the preterm infant's intestines. These effects are correlated with various morbidities of inflammatory origin that are often severe and sometimes fatal. Lower incidences of necrotizing enterocolitis (NEC), retinopathy of prematurity (ROP), sepsis and bronchopulmonary dysplasia have also been demonstrated in preterm infants receiving human milk than for infants exclusively receiving formula milk. Another documented benefit of providing human milk to preterm infants is a shorter time to tolerance of full enteral feedings. Human donor milk is sometimes regarded as a better option than formula milk but availability is limited. It also has to be pasteurized, which compromises nutritional, immunologic, and other milk components.

However, females who have had a preterm delivery encounter significant difficulties when it comes to providing their infants with MoM. In particular, the breast of a preterm female has not yet fully undergone the physiological and morphological changes in preparation for lactation. In addition, having given birth to a preterm infant, a new mother may face additional anxiety and stress that is counterproductive for lactation.

A complete emptying of the breasts is of importance both in the establishment of lactation and its continuation. Otherwise residual milk in the mammary glands will exert a negative feed-back on milk production and lead to an associated involution of mammary tissue. Oxytocin is considered as the major hormone responsible for milk ejection in humans. It has also been shown to increase the secretion and release of prolactin (the major hormone responsible for milk synthesis in animals and humans). However, oxytocin is known to lack selectivity over the vasopressin receptors and so this can lead to side effects when used in the clinic.

Merotocin is a peptidic analogue of oxytocin containing two synthetic amino acid residues. The molecule contains an oxytocin-like 20-membered ring that is closed with a C—S bond between the γ-carbon of the Bua1 residue and the sulphur atom of the Cys6 residue. Two amino acids i.e. 4-Fluoro-benzylglycine and L-1-Carboxypropylcysteine are unnatural.

The chemical structure of merotocin is illustrated below:

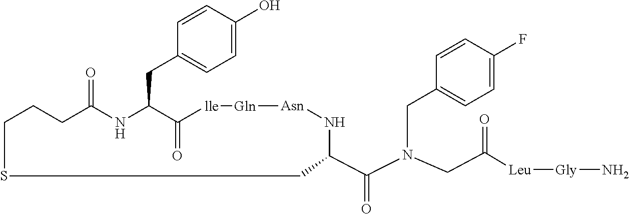

Alternatively, the structure of merotocin may be represented as:

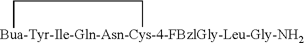

Where Bua is a γ-substituted butyric acid residue and 4-FBzlGly is N-(4-fluorobenzyl)glycine.

Merotocin is also referred to as Glycinamide, N-(4-mercapto-1-oxobutyl)-L-tyrosyl-L-isoleucyl-Lglutaminyl-L-asparaginyl-L-cysteinyl-N-[(4-fluorophenyl)methyl]glycyl-L-leucyl-, cyclic (1→5)-thioether or [Bua¹,4-FBzlGly⁷]oxytocin. Merotocin is also sometimes referred to or designated as compound FE 202767.

WO 2009/122285 (Algarsamy et al) describes a number of peptidic oxytocin receptor agonists including merotocin. This document suggests the use of such compounds in the treatment of compromised lactation conditions and generally describes a dose in the range of 0.05 to 1.0 μg/kg body weight.

Kazimierz Wisniewski et al (Journal of Medicinal Chemistry 2014, 57, 5306-5317) further describe a class of peptidic oxytocin receptor agonists including merotocin and suggest their potential use in the treatment of preterm mothers requiring lactation support.

US 2013/210746 (Siekmann et al) describes a number of aqueous pharmaceutical compositions suitable for nasal administration that have improved stability. In certain examples, the pharmaceutical compositions comprise merotocin.

In addition to the above, US 2012/0214733 (Wisniewski et al) describes a number of other oxytocin receptor agonists and suggests a number of therapeutic uses including lactation induction and maintenance. Further oxytocin analogues were prepared and found to exhibit biological activity in Grzonka et al (Journal of Medicinal Chemistry 1983, 26, 1786-1787). Still further oxytocin analogues are described in WO 2011/120071 (Alewood et al).

Furthermore, US2016/030585 (Barnes et al) describes the use of conjugates comprising a biomolecule linked to a fatty acid to increase the half-life of the biomolecule. In some cases the biomolecule may be an oxytocin receptor agonist. The application describes the potential use of such conjugates in the treatment of a number of conditions including compromised lactation conditions.

### SUMMARY

The present disclosure provides novel doses and treatment regimens of the oxytocin receptor agonist merotocin; these doses and regimens have a number of surprising benefits when used to improve lactation and/or in the treatment or prevention of compromised lactation conditions.

In particular, it has been identified that a composition comprising a relatively high dosage of merotocin can be administered to a female in need thereof to improve lactation. The present inventors have observed that not only does this composition provide significant improvements in lactation but, surprisingly, the high dose of merotocin carries a minimal risk of side effects both in the female and infants fed milk produced following treatment. Indeed, milk produced by females treated with the disclosed compositions shows minimal quantities of merotocin. Accordingly, the doses, compositions and treatment regimens disclosed herein are associated with a reduced risk of (i) systemic exposure to merotocin in any infant fed this milk and (ii) merotocin transfer via breast milk.

Therefore, the present disclosure provides a composition comprising merotocin for use in treating or preventing compromised lactation conditions. The disclosure may also provide a composition comprising merotocin for use in a method of improving lactation in a female in need thereof, wherein the composition is (to be, or intended to be) administered intranasally. The composition comprises a relatively high dose of merotocin that is higher than the dose of oxytocin at which it is expected that vasopressin-mediated side effects can be avoided.

A composition for use may be formulated for intranasal administration and may comprise greater than or at least about 100 μg (e.g. about 400 μg) merotocin. Similarly, a method of treating or preventing a compromised lactation condition or improving lactation in a female in need thereof, may comprise the step of intranasally administering a composition comprising greater than or at least about 100 μg (e.g. about 400 μg) merotocin to the female.

In view of the above, the disclosure further provides the use of at least about 100 μg (e.g. about 400 μg) of merotocin in the manufacture of a medicament for treating or preventing a compromised lactation condition or for improving lactation in a female in need thereof. A medicament for use may be formulated to be administered intranasally.

In some instances, the composition may comprise greater than or at least about 100 μg, 150 μg, 200 μg, 300 μg or 400 μg of merotocin. For example, the composition may comprise between about 100 μg and 500 μg, or between about 200 μg and 450 μg, or between about 300 μg and 400 μg of merotocin. By way of further example, the composition may comprise about 100 μg, 150 μg, 200 μg, 300 μg or 400 μg of merotocin. The amount of merotocin present in the composition may provide a dose of merotocin to be administered to the female. Thus, a dose of merotocin corresponding to any of the amounts above (e.g. 400 μg merotocin per dose) may be used in the compositions, medicaments and methods described herein.

Accordingly, the composition may comprise about 400 μg merotocin for use in a method of improving lactation in a female in need thereof, wherein the composition is administered intranasally.

Indeed, the present inventors have identified that a higher than expected dose of merotocin can be used to improve lactation whilst minimising the risk of side effects suffered by the female. Whilst the use of oxytocin has been suggested to enhance the onset and maintenance of lactation, it is known to lack selectivity over the vasopressin receptors (especially the V2 receptor). Therefore a significant disadvantage of using oxytocin is the risk of vasopressin receptor-mediated side effects (e.g. antidiuresis and hyponatremia).

In particular, in some instances the compositions described herein comprise greater than or at least about 100 μg (e.g. about 400 μg) of merotocin, which is significantly higher than the dose of oxytocin at which it is expected that vasopressin-mediated side effects can be avoided.

Without being bound by theory, it is believed that this higher than expected dose of merotocin can be used in part because merotocin is a selective oxytocin receptor agonist that has low vasopressin receptor activity. Thus, it can be administered at these unexpectedly higher dosages to maximise an improvement in lactation whilst still showing a reduced risk of side effects in the female.

In addition, when looking to provide a treatment of a compromised lactation condition, not only must the treatment be efficacious (e.g. provide an improvement in lactation in the female undergoing the treatment) but there must also be minimal risk to an infant who is fed any milk produced following this treatment.

Advantageously, the present inventors have identified that the intranasal administration of a composition comprising greater than or at least about 100 μg (e.g. about 400 μg) of merotocin not only provides an improvement in lactation in the female but it also results a milk product with minimal quantities of merotocin. Thus, the risk that some quantity of merotocin may become transferred to an infant or that an infant might become exposed to merotocin, is minimised.

Thus, there is also provided a composition, for example a composition, comprising merotocin for use in treating or preventing a compromised lactation condition or for use in improving lactation in a female in need thereof, wherein the composition is intranasally administered and the amount of merotocin present in the milk expressed from the female is minimal, negligible and/or below the lower limit of quantification (LLOQ).

Accordingly, a method of treating or preventing a compromised lactation condition or improving lactation in a female in need thereof is also described, comprising the steps of:
intranasally administering a composition comprising merotocin to the female; and
expressing milk from the female, wherein the amount of merotocin present in the expressed milk is minimal, negligible and/or below the lower limit of quantification (LLOQ).

The method may further comprise feeding the expressed milk to an infant. In some cases, the milk may be expressed by a breast pump or the like. In other cases, the infant may be directly breastfed by the female.

For example, the concentration of merotocin in the milk may be less than 10 μg/mL, less than 5 μg/mL, less than 1 μg/mL, less than 10 ng/mL, less than 5 ng/mL, less than 1 ng/mL, less than 100 μg/mL, less than 50 μg/mL or less than 25 μg/mL. In some instances, the concentration of merotocin in the milk may be less than 25 μg/mL.

Without being bound by theory, the inventors hypothesise that the minimal quantities of merotocin observed in the milk may be attributable to the relatively short half-life of merotocin that means that it is rapidly cleared from the female. In addition, the inventors have identified that merotocin is degradable by chymotrypsin and so the risk to the infant is further minimised.

The compositions, medicaments and methods described herein may be useful in the treatment of any female who is suffering from a compromised lactation condition. Such conditions may be characterised by reduced or inadequate lactation. A female subjected to a method of this disclosure or treated or administered a composition disclosed herein, may, for example, be any post-partum female in need thereof. As used herein, “compromised” “reduced” or “inadequate” lactation” may mean that a female has not yet begun lactating, has a low milk supply and/or is not producing sufficient milk to meet the infant's needs.

By way of example only, a female suffering from a compromised lactation condition may be incapable of producing or expressing a daily volume of milk greater than 50 mL, 100 mL, 200 mL or 400 mL.

In some instances, the female suffering from a compromised lactation condition may be a preterm female. The normal gestation period in a human female is around 40 weeks and an infant is considered as preterm if born prior to 37 weeks gestation. Therefore, as used herein, a “preterm female” may be a female who has delivered an infant prior to 37 weeks gestation. A preterm female may experience additional difficulties when attempting to breastfeed an infant as the breasts of such females have not yet fully undergone the physiological and morphological changes in preparation for lactation.

In some cases, the preterm female may have delivered an infant at a gestational age of between 32 and 37 weeks, e.g. 32 weeks+0 days and 36 weeks+6 days (often referred to as moderate to late preterm). In some cases, the preterm female may have delivered an infant at a gestational age of between 28 and 32 weeks, e.g. 28 weeks+0 days and 31 weeks+6 days (often referred to as very preterm). In other cases, the preterm female may have delivered an infant at a gestational age less than 28 weeks e.g. up to 27 weeks+6 days (often referred to as extremely preterm). The compositions and methods described herein may find particular use in the treatment of preterm females who have delivered an infant at a gestational age between 24 weeks+0 days and 31 weeks+6 days.

As used herein, the term “female” may refer to a mammalian female (e.g. a female capable of lactating). In some cases, the female may be a human female.

As used herein, the term “infant” may refer to the fetus (whether singleton or multiple gestation) or neonate as appropriate.

As is further detailed herein, the administration or use of a disclosed composition or medicament to improve lactation in a female in need thereof (e.g. one suffering from compromised lactation condition) may increase the likelihood of the female producing sufficient milk for an infant and/or establishing a reliable milk supply in both the short-term and long-term.

In particular, the use of the composition in a method of improving lactation may achieve one or more of the following outcomes:
(i) increasing milk production;
(ii) facilitating, stimulating and/or promoting the onset of lactation;
(iii) facilitating and/or promoting the maintenance of lactation; and/or
(iv) increasing the likelihood of lactation.

Where the method, composition or medicament is used to increase milk production, the subject (i.e. a female administered a composition or medicament of this disclosure or a female subject to a method disclosed herein) may produce a greater volume of milk. The greater volume of milk produced by the female may be greater than the volume of milk produced by the same female if she had not been administered a composition or medicament of this disclosure.

The greater volume of milk produced may be assessed over a set period of time. The set period of time may be a period of time following administration of the composition and/or initiation of a treatment regimen comprising multiple administrations of the composition. For example, the set period of time may be up to 1 hour, 2 hours, 3 hours, 4 hours, 5 hours or 6 hours, or up to 1 day, 2 days, 3 days, 4 days, 5 days, 10 days, 14 days, 17 days or 20 days, or up to 1 month, 2 months, 3 months, 6 months or to 12 months following administration of the composition and/or initiation of a treatment regimen comprising a composition of this disclosure.

For example, the female may produce a greater volume of milk in the 1 hour, 2 hours, 3 hours, 4 hours, 5 hours or 6 hours period following administration of the composition and/or initiation of a treatment regimen comprising a composition of this disclosure. By way of further example, the female may produce a greater volume of milk in the 1 day, 2 day, 3 day, 4 day, 5 day, 10 day, 14 day, 17 day or 20 day period following administration of the composition and/or initiation of a treatment regimen comprising a composition of this disclosure. In a yet further example, the female may produce a greater volume of milk in the 1 month, 2 month, 3 month, 6 month or 12 month period following administration of the composition and/or initiation of a treatment regimen comprising a composition of this disclosure.

In some cases, a female administered the composition may produce a greater volume of milk in the 14 day period following initiation of a treatment regimen comprising the composition.

As stated above, improving lactation may comprise facilitating, stimulating and/or promoting the onset of lactation. The onset of lactation following use of a method, composition or medicament of this disclosure may be a first occurrence of lactation in a female following the delivery of an infant; as such, the use of a medicament, method or composition of this disclosure may initiate lactation in the female in need thereof.

Alternatively, facilitating, stimulating, initiating and/or promoting the onset of lactation may mean that a female administered a composition of this disclosure is able to reach a reference or minimum daily volume of milk in a shorter period of time (as compared to the length of time taken to reach the same reference or minimum milk volume in the absence of the use of a composition, method or medicament of this disclosure).

The reference or minimum daily volume of milk may be greater than or equal to 500 mL, or greater than or equal to 750 mL. In other words, a first occurrence of a daily volume of milk produced greater than or equal to 500 mL or 750 mL may be reached more quickly following treatment with a composition of this disclosure.

In some cases, a female may produce a daily volume of milk greater than or equal to 500 mL or 750 mL for the first time on the first day (e.g. within 24 hours) following administration of the composition and/or following initiation of a treatment regimen comprising a disclosed composition. In some cases, a female may produce a daily volume of milk greater than or equal to 500 mL or 750 mL for the first time on any or all of 2, 3, 4, 5, 10, or 14 days following administration of a composition of this disclosure and/or following initiation of a treatment regimen comprising a composition of this disclosure.

As also described above, the methods, uses and medicaments which exploit a composition disclosed herein may facilitate and/or promote the maintenance of lactation. The maintenance of lactation may be assessed by establishing the number of consecutive days on which the reference or minimum daily volume of milk is reached by the female treated using a method, composition or medicament described herein. For example, the number of consecutive days on which the female produces a daily volume of milk greater than or equal to 500 mL or 750 mL may be 2, 3, 4, 5, 10, or 14 days.

The methods, uses, medicaments and compositions may be used to facilitate and/or promote the long-term maintenance of lactation, e.g. even after discontinuation of the treatment. For example, the female may continue to produce a greater volume of milk in the days, weeks and months following the administration of a composition or medicament described herein than she might have done if she had not been administered the composition or medicament. In particular, studies have indicated that interventions promoting the initiation and maintenance of a milk supply during the first week postpartum are important in establishing lactation in the longer term (Hill et al, J. Hum. Lactation, 2005 21: 22-30).

Following the use or administration of a composition, method or medicament of this disclosure and/or initiation of a treatment regimen comprising a composition described herein, the volume of mother's own milk (MoM) fed to an infant may be increased. Alternatively or additionally, the volume of formula or donor milk fed to the infant may be decreased.

As stated, a composition of this disclosure is typically administered intranasally to a female in need thereof. Accordingly, the composition may be adapted for nasal administration.

A composition of this disclosure may be a pharmaceutical composition. A composition of this type may be sterile and/or may further comprise one or more pharmaceutically acceptable excipients, diluents and/or buffers. For convenience, any reference to a “composition” (of this disclosure of disclosed herein) should be understood as also embracing a “pharmaceutical composition”.

A useful composition may comprise a liquid and/or aqueous preparation of merotocin (e.g. a sterile aqueous preparation of merotocin). The preparation may be formulated using suitable diluents, dispersing agents, wetting agents and/or suspending agents. Acceptable diluents for use in the preparation include water, Ringer's solution and isotonic sodium chloride solution. The preparation may additionally comprise excipients such as sodium phosphate, citric acid, sodium chloride, glycerine, sorbitol solution, methylparaben, propylparaben and chlorobutanol.

The preparation may include any of those preparations described in US 2013/0210746 which is herein incorporated by reference.

For instance, a composition of this disclosure may comprise a buffering agent, for example acetic acid, adipic acid, citric acid, maleic acid, succinic acid or (e.g. sodium) phosphate. The composition may include a single buffering agent. The composition may include more than one buffering agent (e.g. may comprise citric acid and (e.g. sodium) phosphate). The composition may comprise a buffer (solution), for example, a citrate buffer (solution), comprising citric acid and a citrate (e.g. sodium citrate); a succinate buffer (solution) comprising succinic acid and a succinate (e.g. sodium succinate), an acetate buffer (solution) comprising acetic acid and an acetate (e.g. sodium acetate); a citrate/phosphate buffer (solution) comprising citric acid and phosphate; or a phosphate buffer (solution).

The concentration of merotocin in the liquid (aqueous) composition may be, for example, from 0.01 to 5 mg/mL. For example, the concentration of merotocin in the liquid (aqueous) composition may be from 0.01 to 4 mg/mL, for example 0.05 to 2 mg/mL, 0.1 to 1.4 mg/mL, or 0.2 to 0.7 mg/mL. In some cases, the concentration of merotocin may be 2 mg/mL.

A composition of this disclosure may further comprise an isotonicity agent. Isotonicity agents, for example, mannitol or NaCl, are well known in the art. The isotonicity agent may be present in an amount sufficient to provide an isotonic composition (solution), for example in an amount of 0.01% to 10% (w/v). The isotonicity agent may be mannitol. If the isotonicity agent is mannitol it may be present in an amount of 0.5% to 7.5% (w/v), more preferably 4.0% to 5.5% (w/v), for example 5.0% (w/v). If the isotonicity agent is mannitol it may be present in an amount of 0.05% to 7.5% (w/v). If the isotonicity agent is NaCl it may be present in an amount of 0.05% to 1.2% (w/v) or 0.08% to 1% (w/v), for example 0.9% (w/v). The isotonicity agent may be present in an amount of 0.1 to 100 mg/mL, for example 0.5 to 7 mg/mL, for example 1 to 5 mg/mL. For example, if the isotonicity agent is mannitol it may be present in an amount of 5 to 75 mg/mL, for example 40 to 55 mg/mL. If the isotonicity agent is NaCl it may be present in an amount of 0.5 to 12 mg/mL, for example 5 to 10 mg/mL. In some cases, the isotonicity agent may be NaCl and it may be present in an amount of about 7.5 mg/mL.

The disclosed compositions may further comprise an anti-oxidant. The anti-oxidant may be any anti-oxidant commonly used in the art, for example any anti-oxidant approved for use as a pharmaceutical excipient. For example, the anti-oxidant may be methionine, EDTA, butylated hydroxy toluene, sodium metabisulfite etc. Preferably the anti-oxidant is present in an amount of 0.01% to 10% (w/v), for example 0.05% to 5% (w/v), most preferably 0.08% to 1% (w/v). Preferably the anti-oxidant is methionine, EDTA, or a combination of methionine and EDTA. In an example, the antioxidant is methionine and is present in an amount of 0.5% w/v. In an example, the antioxidant is EDTA and is present in an amount of 0.1% w/v.

A useful composition may include an enhancer, e.g. an excipient which enhances the effective dose (e.g. enhances the effective dose following nasal administration). The enhancer may be any enhancer commonly used in the art, for example any enhancer approved for use as a pharmaceutical excipient. The enhancer may be, for example, methyl-β-cyclodextrin, Polysorbate 80, carboxymethylcellulose or hydroxypropyl methylcellulose.

The pH of the compositions described herein may be from 5.0 to 6.0. The pH of the composition may be from 5.0 to 5.9, for example from 5.1 to 5.9, for example 5.2 to 5.8. The pH of the composition may be from 5.1 to 6.0, for example from 5.2 to 6, for example from 5.26 to 6. The pH of the composition may be from 5.15 to 5.75, for example from 5.2 to 5.65. The pH of the composition may be from 5.26 to 5.8, for example from 5.26 to 5.75, for example from 5.26 to 5.7, for example from 5.26 to 5.65, for example 5.4 to 5.65, or about pH 5.5. At such pH values, compositions comprising merotocin may provide stable compositions without the requirement of an anti-oxidant. Such compositions may be particularly suitable for nasal administration.

In some instances, the composition may comprise a succinate buffer or a citrate buffer and the pH may be from 5.0 to 5.9, for example 5.0 to 5.8, for example from 5 to 5.7. For example, the composition may comprise a citrate/phosphate buffer and the pH may be from 5.1 to 6.0, for example from 5.2 to 6, for example from 5.26 to 6.

The composition may take the form of a nasal spray. In other words, the composition may be administered to the female by spraying into one or both nostrils.

The composition may be delivered to the female as a single spray or as multiple (e.g. two or more) sprays to one or both nostrils. Where the composition is to be delivered by way of multiple sprays, the dose of merotocin may be divided between each spray to provide multiple (e.g. two or more) sub-doses.

By way of example, a total dose of 400 μg of merotocin may be delivered by way of multiple sub-doses. In this scenario, the dose may be delivered as two sprays (e.g. one spray in each nostril), each spray comprising a sub-dose of 200 μg of merotocin—thus the combined dose of the two sprays would be the required 400 μg total dose of merotocin.

The disclosure also provides a regimen by which a composition or medicament of this disclosure may be used to treat a subject (so as to improve lactation and/or treat or prevent a compromised lactation condition). For example, a female in need thereof (e.g. a female suffering from compromised or reduced lactation) may be administered a composition of this disclosure multiple times over a predetermined period of time.

For example, a method of improving lactation may comprise a treatment regimen in which a composition or medicament of this disclosure is administered to the female each time milk expression is desired or intended.

There is also provided the use of a composition comprising merotocin in the manufacture of a medicament for treating or preventing a compromised lactation condition and/or improving lactation in a female in need thereof, wherein the composition is (to be) administered intranasally each time milk expression is desired or intended.

A composition of this disclosure may be (intended to be) administered to the female before milk expression is desired or intended. For example, the composition may be (or intended to be) administered shortly before a milk expression session (e.g. where milk is expressed using a breast pump or by other means) and/or shortly before a female wishes to breastfeed an infant. In some cases, the composition may be (or may intended to be) administered up to 5, 10, 15, 20 or 30 minutes before a milk expression session or before a breast feed. In some cases, the composition is administered (or is intended to be administered) to the female between about 1 and 20 minutes, 2 and 15 minutes, or 5 and 10 minutes before a milk expression session or a breast feed is desired or intended. In some cases, the composition is administered to the female between about 10 and 20 minutes, or between about 12 and 15 minutes before a milk expression session or a breast feed is desired or intended.

A suitable treatment regimen may comprise the daily administration of a composition of this disclosure to the female in need thereof. For example, a composition may be administered multiple times in a single day. In some cases, the composition may be administered once per day or may be administered anywhere between about 2 and 10 times per day. By way of further example, the composition may be administered between 5 and 10 times, or between 6 and 8 times per day. This treatment may continue for multiple days or weeks.

As explained above, the composition may be administered each time milk expression is desired or intended. For example, the composition may be administered to a female about 1 and 20 minutes, 2 and 15 minutes, 10 and 20 minutes, 12 and 15 minutes or 5 and 10 minutes before milk expression is desired or intended and between 5 and 10 times, or between 6 and 8 times per day.

A composition or medicament of this disclosure may be administered to the female at regular or irregular intervals, e.g. at regular or irregular intervals throughout every 24 hour period. For example, the composition may be administered to the female at intervals anywhere between 1 and 6 hours, or anywhere between 2 and 5 hour intervals. In some cases, no more than 5 hours will elapse between administrations of the composition.

A composition or medicament of this disclosure may be administered to the female within 12 hours of delivery, within 24 hours of delivery, within 48 hours of delivery or within 72 hours of delivery. The composition may be administered to the female following an attempt at initial milk expression within 12 hours of delivery. By way of further example, the composition may be administered to the female provided that milk expression has been attempted multiple times following delivery, e.g. provided that the female has attempted milk expression at least 2, 3, or 4 times every 24 hours following delivery or from 24 hours of delivery.

The female may continue such treatment for as long as she wishes to provide breast milk to the infant. Alternatively, the female may continue such treatment until such time as lactation is fully established.

For instance, in some cases, the treatment may be discontinued once lactation and/or the milk supply has been established in the female. For example, lactation and/or the milk supply may be considered established if the female has expressed at least 500 mL or 750 mL MoM on a number of consecutive days (e.g. 2, 3, 4, 5, 10 or 14 days).

When looking to devise an appropriate treatment regime for improving lactation in a female in need thereof, a number of factors need to be considered. One such factor is that some females may prefer to feed their infants on-demand rather than on a fixed schedule. “On-demand” feeding may also be referred to as “responsive feeding” or “baby-led feeding”. In such cases, the female responds to the infant's hunger cues and initiates feeding when the infant appears hungry.

Therefore, it is important that any treatment is quick-acting so that a female may respond quickly to an infant's hunger cues. It may also be important that the treatment can be used multiple times in a relatively short period of time. For example, many infants will prefer to cluster feed at certain periods of the day (e.g. where an infant wishes to feed more often in a shorter period of time).

The inventors have observed that the use of the various compositions and medicaments of this disclosure does not result in the accumulation of merotocin in the female—this, despite multiple administrations of the composition. Advantageously, this means that the compositions and medicaments described herein can be administered multiple times throughout the day and for extended period of time (including each time milk expression is intended or desired) with minimal risk to the female and/or infant.

In this regard, it has been observed that the half-life (t_{1/2}) and time to maximum plasma concentration (tₘₐₓ) of merotocin were broadly similar across the various doses that were administered to the females intranasally. Without being bound by theory, the inventors believe that absorption is the rate limiting step for the kinetics of merotocin following intranasal administration. In particular, it was observed that the intranasal administration of merotocin provided a tₘₐₓ of approximately 12-15 minutes and a t_{1/2} of between 25 and 35 minutes at a number of different doses. Thus, administering the composition to the female between about 1 and 20 minutes, 10 and 20 minutes, 12 and 15 minutes, 2 and 15 minutes, or 5 and 10 minutes before milk expression is desired or intended may advantageously mean that tₘₐₓ is reached shortly before or during milk expression.

Without being bound by theory, it is hypothesised that the intranasal administration of merotocin is particularly well-suited to assist in the establishment of lactation and/or breastfeeding of an infant. In particular, the relatively short tₘₐₓ may mean that the female can initiate breastfeeding shortly after observing hunger cues from her infant. In addition, the relatively short t_{1/2} may mean that the female can breastfeed an infant multiple times in a relatively short period of time as the risk of merotocin accumulating to dangerous systemic levels is low. Accordingly, a female may be able to respond more effectively to cluster feeding demands from an infant. Having the ability to respond quickly and/or appropriately to an infant's hunger cues at the early stages of lactation and/or breastfeeding may be important in the establishment and long-term continuation of lactation and/or breastfeeding.

It should be understood that, where appropriate, the term “comprising” may encompass the terms “consisting essentially of” and “consisting of”. Additionally, each of the definitions and embodiments provided in this specification applies to each aspect of this disclosure.

### DETAILED DESCRIPTION

The present disclosure will now be further described with reference to the following data:

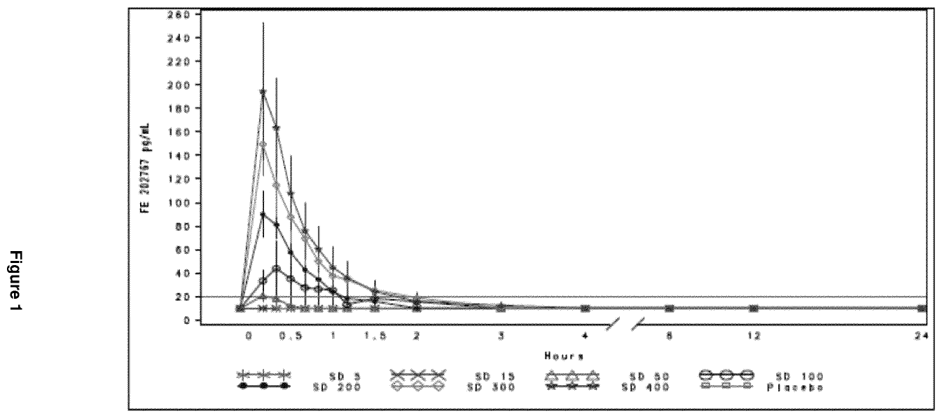

FIG. 1 shows merotocin plasma concentration (mean) after single intranasal administration of 5 μg, 15 μg, 50 rig, 100 μg, 200 μg, 300 μg and 400 μg merotocin and a placebo.

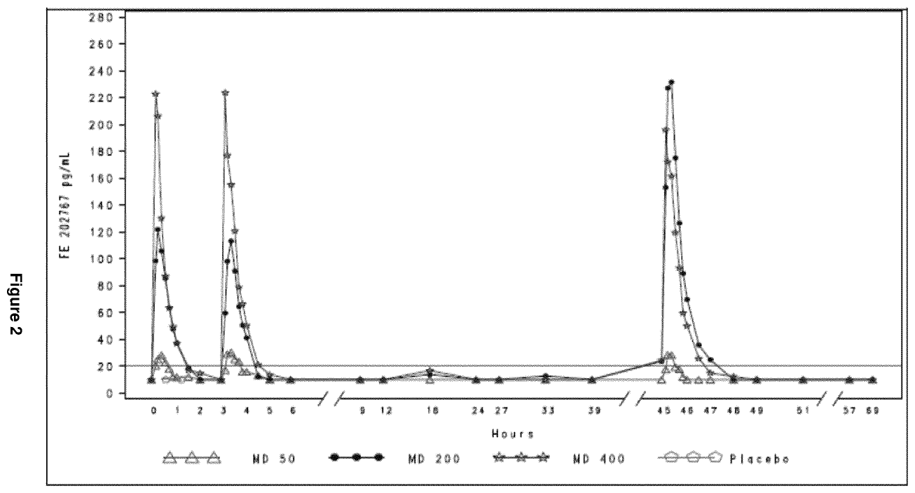

FIG. 2 shows merotocin plasma concentration (mean) after multiple intranasal administrations of 50 μg, 200 μg and 400 μg merotocin and a placebo (16 doses over 49 hours).

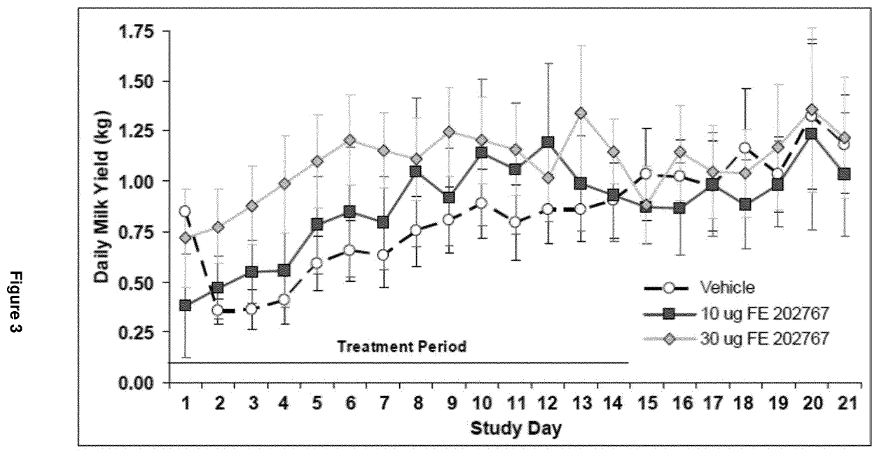

FIG. 3 shows the effect of merotocin on daily milk yield in ewes with induced pre-term delivery. Merotocin (FE 202767) and oxytocin were administered as intramuscular bolus doses. Data are presented as mean±S.E.M.

### CLINICAL PHARMACOLOGY TRIALS

A total of 104 healthy female volunteers, of whom 84 were exposed to merotocin, have been included in two Phase 1 trials: one was a single ascending-dose and multiple ascending dose trial; the other was a milk-transfer trial.

An overview of the trial designs, dose regimens, study populations and key results is presented in Table 1.

TABLE 1
Overview of the clinical studies

| | | Investigational | | |
|---|---|---|---|---|
| Study | | drug | | |
| code and | Study | Dosage | Study | Key |
| objective | design | regimen/route | population | results |
| Trial | Double-blind | 5-400 μg | Healthy | No safety |
| 000015 | (partially), | merotocin | women | concerns |
| First in | placebo | single dose i.n. | N = 86, | tmax ≈ 15 min, |
| human | controlled, | 50, 200, 400 μg | 66 active | t½ ≈ 30 min |
| | sequential | merotocin | treatment, | after i.n. |
| | dose | every 3 hours | 20 placebo | administration |
| | escalation, | for 45 hours i.n. | | |
| | within | 20 μg i.v., | | |
| | dose | 400 μg i.n. | | |
| | randomised | merotocin | | |
| | | cross-over | | |
| Trial | Open label | 5 and 20 μg i.v. | Healthy | No milk |
| 000028 | | | women | transfer |
| Milk | | | 5 μg: | detectable |
| transfer | | | N = 6 | No safety |
| | | | 20 μg | concerns |
| | | | N = 12 | |

Trial 000015 (Safety and Pharmacokinetics after Single and Multiple Intranasal and Single Intravenous Doses in Healthy Women)

The first clinical trial in human, Trial 000015, was a double-blind, placebo-controlled, single dose, within dose panel randomised, sequential dose escalating study. The trial was divided into three parts:

Part 1. Ascending single i.n. doses. There were 7 dose panels with 8 healthy women in each dose panel (6 active, 2 placebo). The doses investigated were 5 μg, 15 μg, 50 μg, 100 μg, 200 μg, 300 μg, and 400 μg.

The subjects in Part 1 of the trial received a single intranasal administration of either FE 202767 or placebo. Seven dose levels were investigated: 5, 15, 50, 100, 200, 300, and 400 μg. Each of the doses were administered as a nasal spray based on one or multiple puffs per nostril.

Part 2. Ascending multiple i.n. doses. There were 3 dose panels with 8 healthy women in each dose panel (6 active, 2 placebo). The dosing schedule was every 3 hours for 45 hours, i.e. 16 doses in all. The doses investigated were 50 μg, 200 μg, and 400 μg.

The subjects in Part 2 of the trial received up to 16 intranasal administrations 3 hours apart of either FE 202767 or placebo. Three dose levels were investigated: the first dose was 50 μg, the second dose was 200 μg (intermediate between the first dose and the maximal tolerated dose in Part 1), and the highest dose was 400 μg (the maximal dose in Part 1).

Part 3. Open-label cross-over single i.n. and single i.v. dose in 6 healthy women. The doses investigated were 20 μg as an i.v. infusion over 25 minutes, and 400 μg as a single i.n. dose.

The subjects in Part 3 of the trial received a single intranasal administration and a single intravenous infusion of FE 202767 with a wash-out period in between. The intranasal dose was 400 μg (4×140 μl, the same as the highest dose in Part 1), the i.v. dose was 20 μg infused in 2 mL over 25 minutes.

The intravenous dose and infusion rate were determined from the intranasal pharmacokinetic data in Part 1. The infusion was performed so that the plasma concentration of FE 202767 resembled the absorption pattern of the corresponding intranasal administration and adjusted in order not to exceed the maximum plasma concentration of, or exposure to, FE 202767 in Part 1. The duration of the infusion was chosen to 25 minutes, which was not less than the tₘₐₓ observed in Part 1 of the trial, and the dose, 20 μg, based on an assumed bioavailability of 5%.

The main objectives were to assess the safety, tolerability, and pharmacokinetics of merotocin, and to estimate the highest tolerable dose.

Trial 000028 (Milk Transfer)

Healthy women received 5 or 20 μg merotocin i.v. shortly after delivery (Part A) or 20 μg merotocin i.v. when lactation was established (Part B). Milk was expressed by pumping at 15 minutes, 3 hours, 6 hours, 9 hours, 12 hours, 18 hours and 18-24 hours after start of infusion and analysed for merotocin and metabolites.

Identity of Investigational Medicinal Product

FE 202767 was provided as an isotonic citrate/phosphate buffered solution of pH 5.5 in water in vials containing an extractable volume of 0.9 mL (Table 2). The concentration of FE 202767 was 0.7 mg/mL, to be used appropriately diluted with buffer or, for Part 3, NaCl for injection. The buffer used for FE 202767 was used as placebo in this study.

TABLE 2
Composition

| Component | Amount per mL |
|---|---|
| FE 202767 | 0.7 mg/mL |
| Sodium phosphate dibasic dihydrate | 3.24 mg/mL |
| Citric acid monohydrate | 1.43 mg/mL |
| Sodium chloride | 7.50 mg/mL |
| NaOH/HCl | To pH 5.5 |

Pharmacokinetics

Single Intranasal Dose

The mean plasma concentration curves are illustrated in FIG. 1 and the pharmacokinetic parameters in healthy women administered a single intranasal (i.n.) dose are presented in Table 3. For the doses 5-50 μg the plasma concentrations above the LLOQ were too few for meaningful calculations of AUC and t_{1/2}, as was the case for AUC and t1/2 with 100 μg. The tₘₐₓ and terminal half-lives were similar in all dose groups, with a tₘₐₓ of 12-15 minutes and a t1/2 of 25-35 minutes. Increasing dose increased the exposure by means of AUC and Cₘₐₓ in a close to dose proportional manner, albeit with substantial inter-individual variation.

TABLE 3
Pharmacokinetic parameters of merotocin after single intranasal administration (Trial 000015)

| | | 100 μg | 200 μg | | 300 μg | | 400 μg | |
|---|---|---|---|---|---|---|---|---|
| | | N = 6 | N = 6 | | N = 6 | | N = 6 | |
| AUCₜ | Geom. mean (% CV) | ND | 35.7 | (59%) | 77.8 | (72%) | 88.6 | (89%) |
| [h*pg/mL] | Median | | 52.5 | | 94.3 | | 119 | |
| Cₘₐₓ | Geom. mean (% CV) | 37.9 (106) | 84.1 | (43) | 143 | (41) | 153 | (68) |
| [pg/mL] | Median | 32.2 | 99.9 | | 148 | | 199 | |
| tₘₐₓ | Mean (SD) | 0.25 (0.09) | 0.25 | (0.09) | 0.20 | (0.07) | 0.22 | (0.08) |

| | | | | | |
|---|---|---|---|---|---|
| [h] | Median | 0.25 | 0.25 | 0.17 | 0.17 |
| t_{1/2} | Harmonic mean | ND | 0.43 | 0.43 | 0.56 |
| [h] | Median | | 0.39 | 0.48 | 0.55 |

N = number of subjects;
ND = not determined

Multiple Intranasal Doses

The mean plasma concentration curves are illustrated in FIG. 2, and the pharmacokinetic parameters in healthy women administered multiple i.n. doses are presented in Table 4. For the 50 μg dose, the plasma concentrations above the LLOQ were too few for meaningful calculations of AUC and t1/2. The tₘₐₓ and t_{1/2} were similar in all dose groups, with a tₘₐₓ of 12-15 minutes and a t_{1/2} of 25-35 minutes, consistent with the observations after single dose administration.

There were no indications of relevant accumulation during the 16 administrations over 48 hours, as demonstrated by the pre-dose concentrations falling constantly below the LLOQ. The AUC and Cₘₐₓ after the last 200 μg dose were unexpectedly high, the pre-dose sample did not indicate that this was due to accumulation.

TABLE 4
Pharmacokinetic parameters of merotocin after multiple
intranasal administrations (Trial 000015)

| | | Dose | 50 μg | | 200 μg | | 400 μg | |
|---|---|---|---|---|---|---|---|---|
| | | No. | N = 6 | | N = 6 | | N = 6 | |
| AUCₜ | Geom. mean (% CV) | 1 | ND | | 80.4 | (33%) | 77.1 | (104%) |
| [h*pg/mL] | Median | | | | 87.6 | | 70.2 | |
| | Geom. mean (% CV) | 2 | ND | | 75.2 | (28%) | 88.8 | (75%) |
| | Median | | | | 70.6 | | 142 | |
| | Geom. mean (% CV) | 16 | ND | | 160 | (61%) | 112 | (71%) |
| | Median | | | | 147 | | 112 | |
| Cₘₐₓ | Geom. mean (% CV) | 1 | 27.5 | (52%) | 124 | (27%) | 156 | (114%) |
| [pg/mL] | Median | | 30.7 | | 125 | | 136 | |
| | Geom. mean (% CV) | 2 | 26.0 | (89%) | 115 | (25%) | 169 | (107%) |
| | Median | | 30.2 | | 118 | | 195 | |
| | Geom. mean (% CV) | 16 | 23.6 | (93%) | 218 | (54%) | 180 | (56%) |
| | Median | | 26.6 | | 225 | | 192 | |
| tₘₐₓ | Mean (SD) | 1 | 0.23 | (0.09) | 0.18 | (0.08) | 0.11 | (.05) |
| [h] | Median | | 0.17 | | 0.17 | | 0.08 | |
| | Mean (SD) | 2 | 0.34 | (0.24) | 0.28 | (0.08) | 0.16 | (0.09) |
| | Median | | 0.25 | | 0.33 | | 0.15 | |
| | Mean (SD) | 16 | 0.34 | (0.24) | 0.31 | (0.12) | 0.30 | (0.21) |

| | Median | | 0.25 | 0.33 | 0.33 |
|---|---|---|---|---|---|
| t_{1/2} | Harmonic mean | 1 | ND | 0 43 | 0.35 |
| [h] | Median | | | 0.43 | 0.43 |
| | Harmonic mean | 2 | ND | 0.44 | 0.39 |
| | Median | | | 0.48 | 0.48 |
| | Harmonic mean | 16 | NF | 0.51 | 0.56 |
| | Median | | | 0.47 | 0.65 |

N = number of subjects;
ND = not determined

Simile Intranasal and Intravenous Cross-Over Doses

The pharmacokinetic parameters in healthy women administered a single intravenous (i.v.) dose of 20 μg infused over 25 minutes and a single 400 μg i.n. dose are presented in Table 5. The pharmacokinetic parameters after the i.n. administration were similar to the single dose pharmacokinetic parameters found in the preceding part of the trial, both with respect to exposure and Cₘₐₓ, and to tₘₐₓ and t_{1/2}. However, the t_{1/2} after i.v. administration was considerably shorter compared to i.n. administration, indicating that the absorption was the rate limiting step for the kinetics of merotocin after i.n. administration. The bioavailability was calculated to 3.7% (range 2.2-6.2%), similar to what is found with other peptides of comparable size.

TABLE 5
Pharmacokinetic parameters of merotocin after a single intranasal
administration and a single intravenous infusion (Trial 000015)

| | | 400 μg i.n. | | 20 μg i.v. | |
|---|---|---|---|---|---|
| | | N = 6 | | N = 6 | |
| AUCₜ | Geom. mean (% CV) | 83.0 | (38%) | 146 | (25%) |
| [h*pg/mL] | Median | 72.8 | | 150 | |
| Cₘₐₓ | Geom. mean (% CV) | 151 | (43%) | 463 | (11) |
| [pg/mL] | Median | 156 | | 463 | |
| tₘₐₓ | Mean (SD) | 0.11 | (0.05) | 0.36 | (0.05) |

| [h] | Median | 0.08 | 0.33 | |
|---|---|---|---|---|
| t_{1/2} | Harmonic mean | 0.43 | 0.17 | |
| [h] | Median | 0.61 | 0.18 | |
| CL | Mean (SD) | — | 134 | (37) |

| | | |
|---|---|---|
| [L/h] | Median | 128 |

| | | | | |
|---|---|---|---|---|
| V_{z} | Mean (SD) | — | 37.5 | (22.6) |

| | | | | |
|---|---|---|---|---|
| [L] | Median | | | 31.2 |
| F | Mean (SD) | 3.7 | (1.5) | — |

| | | |
|---|---|---|
| [%] | Median | 3.4 |

N = number of subjects

Metabolism

Plasma and urine samples from Trial 000015 were investigated for the presence of metabolites of merotocin. No metabolites of merotocin could be detected in the plasma or urine samples analysed.

Milk Transfer

None of the milk samples contained merotocin concentration above the LLOQ, 25 μg/mL. The mean total amount of milk collected was about 500-700 mL over 24 hours in the various groups studied.

Clinical Safety—Adverse Events

Single Dose Administration

In Trial 000015, 48 treatment-emergent AEs (TEAEs) were reported by 30 (44%) of the 68 subjects (62 unique subjects) administered a single dose of merotocin or placebo (subjects receiving both i.v. and i.n. administration are regarded as separate treatments and are counted in both groups) (Table 6). All TEAEs except those in the 50 μg or 100 μg groups were assessed by investigators to be related to IMP (i.e. adverse drug reactions (ADRs)). The most frequently reported TEAEs were headache (reported by 19 subjects and in all treatment groups), flushing or hot flush (reported by 6 subjects, 4 of whom in the 20 μg i.v. group), and dizziness (reported by 4 subjects, 2 of whom were in the placebo group). Dry mouth was reported in 3 subjects receiving merotocin. Tachycardia or sinus tachycardia were reported in 3 subjects in the 20 μg i.v. group. Other TEAEs were reported by only 1 or 2 subjects.

All adverse events after single dose administration were regarded as mild.

TABLE 6
Treatment-emergent adverse events after single administration in Trial 000015.

| | 5 μg i.n. | 15 μg i.n. | 50 μg i.n. | 100 μg i.n. | 200 μg in. | 300 μg i.n. | 400 μg i.n. | 20 μg i.v. | Placebo |
|---|---|---|---|---|---|---|---|---|---|
| System Organ Class | N = 6 | N = 6 | N = 6 | N = 6 | N = 6 | N = 6 | N = 12 | N = 6 | N = 14 |
| Preferred Term | n (%) E | n (%) E | n (%) E | n (%) E | n (%) E | n (%) E | n (%) E | n (%) E | n (%) E |
| Any AE | 3 (50) 5 | 2 (33) 2 | 2 (33) 3 | 1 (17) 1 | 4 (67) 7 | 1 (17) 2 | 6 (50) 7 | 6 (100) 11 | 5 (36) 10 |

Cardiac disorders

| | |
|---|---|
| Sinus | 1 (17) 1 |
| tachycardia | |
| Tachycardia | 2 (33) 2 |

Nervous system disorders

| | | | | | | | | | |
|---|---|---|---|---|---|---|---|---|---|
| Headache | 2 (33) 2 | 1 (17) 1 | 2 (33) 3 | 1 (17) 1 | 3 (50) 3 | 1 (17) 1 | 4 (33) 4 | 2 (33) 2 | 3 (21) 3 |
| Dizziness | | | | | | | 1 (8) 1 | 1 (17) 1 | 2 (14) 2 |
| Dysgeusia | | | | | 2 (33) 2 | | | | |

Gastrointestinal disorders

| | | | |
|---|---|---|---|
| Dry mouth | 2 (33) 2 | 1 (17) 1 | |
| Nausea | 1 (17) 1 | | 1 (7) 1 |

Reproductive system and breast disorders

| | | |
|---|---|---|
| Breast | 1 (17) 1 | 1 (7) 1 |
| tenderness | | |
| Breast | | 1 (7) 1 |
| discharge | | |

Skin and subcutaneous tissue disorders

| | | |
|---|---|---|
| Hyperhidrosis | 1 (17) 1 | 1 (7) 2 |

Ear and labyrinth disorders

| | |
|---|---|
| Vertigo | 1 (17) 1 |

Respiratory, thoracic, and mediastinal disorders

| | |
|---|---|
| Intranasal | 1 (17) 1 |
| hypoasthesia | |

Vascular disorders

| | | | |
|---|---|---|---|
| Flushing | 1 (17) 1 | 1 (8) 1 | 3 (50) 3 |
| Hot flush | | | 1 (17) 1 |

AE = adverse event, N = Number of subjects in the Safety Analysis Set, n = Number of subjects with AE, % = Proportion of subjects in the analysis having an AE, E = Number of AEs.

Treatment-emergent AEs in early postpartum women participating in the milk transfer study (Trial 000028) and receiving a single dose of merotocin by i.v. administration are summarized in Table 7. No severe or serious AE occurred, and no AE lead to death or discontinuation from the study. The most frequently reported TEAEs were uterine spasm (in both the 5 μg and 20 μg dose groups), breast engorgement (in the 20 μg dose group), and headache (in the 5 μg dose group). All TEAEs in the trial were of mild intensity, with the exception of 1 event of uterine spasm of moderate intensity reported in the 5 μg dose group in Part A. All TEAEs in the trial, except 1 event of muscle spasm reported in the 5 μg dose group in Part A, were regarded as ADRs by investigators.

TABLE 7
Treatment-emergent adverse events by system organ class
and preferred term after a single intravenous infusion
in early postpartum women in Trial 000028.

| | Part A | Part A | Part B | |
|---|---|---|---|---|
| | 5 μg | 20 μg | 20 μg | Total |
| System Organ Class | (N = 6) | (N = 6) | (N = 6) | (N = 18) |
| Preferred Term | n (%) E | n (%) E | n (%) E | n (%) E |
| Any AE | 3 (50) 9 | 5 (83) 7 | 0 | 8 (44) 16 |

Reproductive system and breast disorders

| | | | | |
|---|---|---|---|---|
| Uterine spasm | 3 (50) 4 | 3 (50) 3 | — | 6 (33) 7 |
| Breast engorgement | | 3 (50) 3 | — | 3 (17) 3 |

Musculoskeletal and connective tissue disorders

| | | | | |
|---|---|---|---|---|
| Back pain | 1 (17) 1 | — | — | 1 (6) 1 |
| Muscle spasms | 1 (17) 1 | — | — | 1 (6) 1 |

Nervous system disorders

| | | | | |
|---|---|---|---|---|
| Headache | 2 (33) 3 | — | — | 2 (11) 3 |

Pregnancy, puerperium and perinatal conditions

| | | | | |
|---|---|---|---|---|
| Lactation puerperal increased | — | 1 (17) 1 | — | 1 (6) 1 |

AE = adverse event, N = Number of subjects in the Safety Analysis Set, n = Number of subjects with AE, % = Proportion of subjects in the analysis having an AE, E = Number of AEs.

Single i.v. administration of 5 and 20 μg merotocin to early postpartum women and 20 μg to women with established lactation were safe and well tolerated as assessed by AEs, vital signs, ECG, and clinical laboratory measurements. All AEs, except one event of moderate intensity, were of mild intensity. No serious adverse event or death occurred.

Multiple Dose Administration

A total of 14 TEAEs were reported by 10 (42%) of the 24 subjects administered multiple doses of merotocin in Trial 000015 (Table 8). All of these TEAEs were regarded as ADRs by investigators.

The most frequently reported AEs were headache (reported by 4 subjects in all treatment groups) and dry mouth (reported by 5 subjects in the 200 and 400 μg groups). Other TEAEs were single observations among all treatment groups. All TEAEs after multiple dose administrations were regarded as mild.

One subject was withdrawn from the 400 μg group due to nausea. The event was regarded as mild, and the subject recovered.

TABLE 8
Treatment-emergent adverse events reported after multiple
intranasal administrations in Trial 000015.

| | 50 μg i.n. | 200 μg i.n. | 400 μg i.n. | |
|---|---|---|---|---|
| System Organ Class | N = 6 | N = 6 | N = 6 | Placebo |
| Preferred Term | n (%) E | n (%) E | n (%) E | N = 12 |
| Any AE | 3 (50%) 3 | 4 (67%) 5 | 3 (50%) 6 | 0 |

Gastrointestinal disorders

| | | |
|---|---|---|
| Dry mouth | 3 (50%) 3 | 2 (33%) 2 |
| Nausea | | 1 (17%) 1 |

Nervous system disorders

| | | | |
|---|---|---|---|
| Headache | 2 (33%) 2 | 1 (17%) 1 | 1 (17%) 2 |

Vascular disorders

| | | |
|---|---|---|
| Flushing | 1 (17%) 1 | 1 (17%) 1 |

Reproductive system and breast disorders

| | |
|---|---|
| Breast tenderness | 1 (17%) 1 |

AE = adverse event, N = Number of subjects in the Safety Analysis Set, n = Number of subjects with AE, % = Proportion of subjects in the analysis having an AE, E = Number of AEs.

### Overview and Conclusions

A single-ascending dose, multiple-ascending dose Phase 1 trial has been conducted in healthy women. The mean Cₘₐₓ after single dose i.n. administration in the highest dose group was 151 μg/mL, and 463 μg/mL after a single i.v. infusion of 20 μg over 25 minutes. The tₘₐₓ and t_{1/2} was approximately 12-15 minutes and 25-35 minutes, respectively, similar at all i.n. dose levels and irrespective of single or multiple administration.

With i.n. administration, the absorption is the rate limiting process controlling the kinetics of merotocin. The most frequently reported AEs were headache, dry mouth and flushing or hot flush. The occurrence of headache in the placebo group was not different from the active treatment groups, while dry mouth and flushing or hot flush were recorded after active treatment only. All AEs were regarded as mild.

The possible transfer of merotocin from plasma to the breast milk was investigated in early postpartum women and in women with established lactation. Following i.v. administration of 20 μg merotocin, corresponding to an i.n. dose of 400 μg, merotocin concentration was below LLOQ, 25 μg/mL, in all milk samples collected up to 24 hours. In early postpartum women, the most frequently reported AEs were uterine spasm, breast engorgement, and headache.

Only occasional events of blood pressure shifts were recorded. High concentrations of merotocin appeared to increase the pulse rate. The ECG and safety laboratory parameters were not affected to any measurable extent by merotocin, and there were no signs of urinary retention at any dose level.

Effect of Merotocin on Milk Production in Pre-Term Sheep

The effect of merotocin on milk production was also investigated in ewes that were induced to deliver pre-term. The ewes were administered vehicle or merotocin intramuscularly twice daily for 14 consecutive days, 10 minutes before morning and evening milking. Merotocin was administered at 10 and 30 μg/animal. The milk volume was measured and the lactose, protein, and fat content analysed.

The 30 μg merotocin treatment group showed an increased daily milk yield relative to vehicle during the first week of treatment, which gradually decreased during the second week of treatment (see FIG. 3). The daily milk yield in the 10 μg merotocin treatment group was only marginally greater compared to the vehicle group during the whole two weeks of treatment.

The total milk yield over the treatment period increased by 27% and 62% in the 10 and 30 μg merotocin groups, respectively. No relevant changes were seen in the lactose, protein, or fat content of the milk.

### LIST OF ABBREVIATIONS AND DEFINITION OF TERMS

AUC area under the plasma concentration-time curve

ADR adverse drug reaction

Cmax maximum plasma concentration

CHO Chinese hamster ovary

CL clearance

CYP cytochrome P450

EC50 drug concentration producing 50% of the maximal effect

ED50 drug dose producing 50% of the maximal effect

Emax maximal response

HEK human embryonic kidney

hERG human ether-á-go-go gene

hOTR human oxytocin receptor

hV1a(R) human vasopressin 1a (receptor)

hV1b(R) human vasopressin 1b (receptor)

hV2(R) human vasopressin 2 (receptor)

IC50 concentration producing 50% of the maximal inhibition

i.n. intranasal(ly)

i.v. intravenous(ly)

IMP Investigational Medicinal Product

INN international nonproprietary name

Ki binding affinity

LLOQ lower limit of quantitation

MFD maximum feasible dose

MTD maximum tolerated dose

NK2 neurokinin A

NOAEL no observed adverse effect level

PK pharmacokinetics

PND post-natal day

s.c. subcutaneous(ly)

TEAE treatment-emergent adverse event

tmax time to maximum plasma concentration

t1/2 terminal half-life

1-26. (canceled)

27. A method of treating or preventing compromised lactation conditions or improving lactation in a female in need thereof, comprising intranasally administering to the female a dose of merotocin of at least 100 μg.

28. The method of claim 27, comprising intranasally administering a dose of merotocin selected from at least 200 μg, at least 300 μg, and at least 400 μg.

29. The method of claim 27, wherein the female is a preterm female.

30. The method of claim 27, comprising administering the dose of merotocin by way of two or more sub-doses.

31. The method of claim 27, comprising administering the dose of merotocin by way of multiple sprays, wherein the total dose administered is divided between each spray to provide multiple sub-doses of merotocin.

32. The method of claim 31, wherein the total dose is 400 μg of merotocin and is delivered by way of two or more sub-doses.

33. The method of claim 27, comprising administering the merotocin at a time selected from up to 5 minutes, up to 10 minutes, up to 15 minutes, up to 20 minutes, and up to 30 minutes, each before a milk expression session or before a breast feed.

34. The method of claim 27, comprising administering the merotocin according to a regimen selected from once per day, 2 to 10 times per day, 5 to 10 times per day, and 6 and 8 times per day.

35. The method of claim 27, comprising administering the merotocin to the female according to a regimen selected from about 1 to 20 minutes, 10 to 20 minutes, 12 to 15 minutes, 2 to 15 minutes, and 5 to 10 minutes, each before milk expression is desired or intended, and for a number of times per day selected from 5 to 10 times and 6 to 8.

36. The method of claim 27, comprising administering the merotocin to the female each time milk expression is desired or intended.

37. The method of claim 27, comprising administering the merotocin to the female at intervals selected from 1 to 6 hour intervals and 2 to 5 hour intervals.

38. The method of claim 27, comprising administering the merotocin to the female at a time selected from within 12 hours of delivery, within 24 hours of delivery, within 48 hours of delivery, and within 72 hours of delivery.

39. The method of claim 27, comprising administering the merotocin to the female following an attempt at initial milk expression within 12 hours of delivery.

40. The method of claim 27, comprising administering the merotocin to the female after milk expression has been attempted multiple times following delivery.

41. The method of claim 27, wherein the amount of merotocin present in milk expressed from the female following administration of the merotocin is at a level selected from minimal, negligible, and below the lower limit of quantification (LLOQ).

42. The method of claim 41, wherein the concentration of merotocin in milk expressed from the female is selected from less than 50 μg/mL and less than 25 μg/mL.

43. The method of claim 27, wherein the method is effective for achieving one or more of the following outcomes:
(i) increasing milk production;
(ii) facilitating, stimulating and/or promoting the onset of lactation;
(iii) facilitating and/or promoting the maintenance of lactation; and
(iv) increasing the likelihood of lactation.

44. The method of claim 27, wherein the female produces a greater volume of milk during a period following administration of the merotocin selected from 1 hour, 2 hours, 3 hours, 4 hours, 5 hours, 6 hours, 1 day, 2 days, 3 days, 4 days, 5 days, 10 days, 14 days, 17 days, and 20 days.

45. The method of claim 27, wherein the female produces a greater volume of milk during a period following initiation of a treatment regimen comprising the merotocin selected from 1 hour, 2 hours, 3 hours, 4 hours, 5 hours, 6 hours, 1 day, 2 days, 3 days, 4 days, 5 days, 10 days, 14 days, 17 days, and 20 days.

46. The method of claim 27, wherein the female produces for the first time a daily volume of milk selected from greater than or equal to 500 mL and greater than or equal to 750 mL, on any or all of days 1, 2, 3, 4, 5, 10, and 14 following administration of the merotocin.

47. The method of claim 27, wherein the merotocin is administered in an aqueous preparation of merotocin.

48. The method of claim 47, wherein the aqueous preparation of merotocin has one or more of the following features:
(i) a citrate/phosphate buffer;
(ii) a concentration of merotocin between 0.05 mg/mL to 2 mg/mL;
(iii) sodium chloride (NaCl); and
(iv) a pH of from 5.2 to 6.
